(19) United States
(12) Patent Application Publication
(10) Pub. No.: US 2013/0079633 A1
(43) Pub. Date: Mar. 28, 2013

(54) Diagnostic System with Hybrid Cable Assembly

(71) Applicants: Jan Peeters Weem, Forest Grove, OR (US); Laurence A. Daane, Portland, OR (US); Jason M. Woods, Portland, OR (US)

(72) Inventors: Jan Peeters Weem, Forest Grove, OR (US); Laurence A. Daane, Portland, OR (US); Jason M. Woods, Portland, OR (US)

(73) Assignee: Tyco Electronics Corporation, Berwyn, PA (US)

(21) Appl. No.: 13/243,957

(22) Filed: Sep. 23, 2011

(51) Int. Cl. A61B 8/00; G02B 6/44; G02B 6/36

(52) U.S. Cl. 600/437; 385/89; 385/101

(57) ABSTRACT

A diagnostic system includes a remote unit configured to gather information and a base unit configured to process the gathered information. A cable couples the remote unit to the base unit and is configured to carry the information. The cable includes one or more electrical conductors for communicating electrical signals between the base unit and the remote unit. The cable also includes one or more optical fibers over which the gathered information is communicated. An outside sleeve is formed around the one or more electrical conductors and the one or more optical fibers.

### BACKGROUND

Many medical devices include a base unit and a remote unit where the remote unit communicates information to and from the base unit. The base unit then processes information communicated from the remote unit and provides diagnostic information, reports, and the like. In some arrangements, a cable that includes a group of electrical wires couples the remote unit to the base unit. The size of the cable typically depends on the number of conductors running through the cable and the gauge or thickness of the conductors. The number of conductors running within the cable tends to be selected according to the amount of information communicated from the remote unit to the base unit. That is, the higher the data rate, the greater the number of conductors.

In more advanced medical devices that use the base/remote unit arrangement, a great deal of information may be communicated between the remote component and the base unit. For example, a transducer of an ultrasound machine may communicate analog information over hundreds of conductors to an ultrasound image processor. This, however, tends to increase the thickness of the cable, making the remote unit somewhat cumbersome to use. To alleviate this problem, higher gauge conductors (i.e., thinner) may be utilized. However, the thinner conductors tend to be more fragile and difficult to handle and terminate.

### BRIEF SUMMARY

An object of the application is to provide a diagnostic system that includes a remote unit configured to gather information and a base unit configured to process the gathered information. A cable couples the remote unit to the base unit and is configured to carry the information. The cable includes one or more electrical conductors for communicating electrical signals between the base unit and the remote unit. The cable also includes one or more optical fibers over which the gathered information is communicated. A common outside jacket contains the electrical conductors and the optical fibers.

Another object of the application is to provide a method for communicating information between a base unit and a remote unit of a diagnostic system. The method includes coupling the base unit to the remote unit with a cable. The cable includes one or more electrical conductors configured to communicate electrical signals between the base unit and the remote unit. The cable also includes one or more optical fibers for communicating optical signals between the base unit and the remote unit. A common outside jacket contains the electrical conductors and the optical fibers. First information is communicated from the base unit to the second unit over the electrical conductors and second information is communicated from the remote unit to the base unit over the optical fibers.

Yet another object is to provide a cable for coupling a base unit to a remote unit for communicating signals between the base unit and the remote unit. The cable includes one or more electrical conductors for communicating electrical signals between the base unit and the remote unit. The cable also includes one or more optical fibers for communicating optical signals between the base unit and the remote unit. A common outside jacket contains the electrical conductors and the optical fibers.

Another object is to provide a method for manufacturing a cable for communicating signals between a base unit and a remote unit. The method includes providing one or more electrical conductors for communicating electrical signals between the base unit and the remote unit and providing one or more optical fibers for communicating optical signals between the base unit and the remote unit. A common outside jacket contains the electrical conductors and the optical fibers.

Other features and advantages will be, or will become, apparent to one with skill in the art upon examination of the following figures and detailed description. It is intended that all such additional features and advantages included within this description be within the scope of the claims, and be protected by the following claims.

### BRIEF DESCRIPTION OF THE DRAWINGS

The accompanying drawings are included to provide a further understanding of the claims, are incorporated in, and constitute a part of this specification. The detailed description and illustrated embodiments described serve to explain the principles defined by the claims.

FIG. 1 is an exemplary system that includes a remote unit that communicates with a base unit via a cable;

FIG. 2 is a cross-sectional view of an exemplary cable that may be utilized in the system of FIG. 1;

FIG. 3 is an exemplary connector assembly that may be attached to an end of the cable of FIG. 2; and

FIG. 4 is an exemplary block diagram of operations that may be performed by the system of FIG. 1.

### DETAILED DESCRIPTION OF THE INVENTION

The embodiments described below overcome the problems with existing base/remote unit systems by providing a hybrid cable for communicating information between a base unit and a remote unit. Generally, the cable includes one or more electrical conductors for communicating power and low data rate information, such as configuration information, from the base unit to the remote unit. High data rate information, such as image data, is communicated from the remote unit to the base unit via one or more optical fibers of the cable.

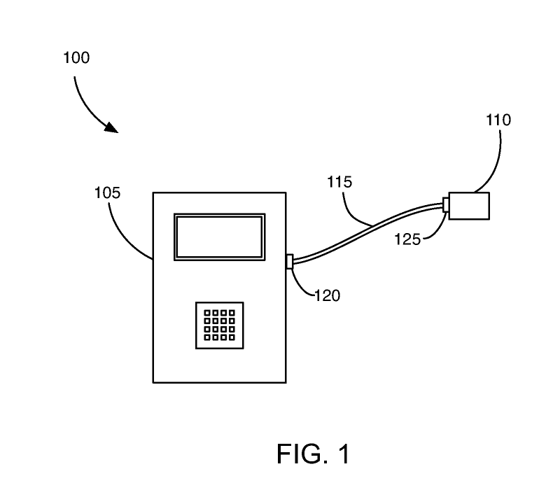

FIG. 1 is an exemplary diagnostic system 100, such as those used in the medical industry. The system 100 includes a base unit 105 and a remote unit 110. The base unit 105 and the remote unit 110 communicate to one another via a cable 115.

The system 100 may correspond to an ultrasound machine. In this regard, the remote unit 110 may correspond to the transducer end of the ultrasound machine and the base unit 105 may correspond to the ultrasound image processor. Power and low data rate information (e.g., lower than about 1 Gb/sec) may be communicated from the base unit 105 to the remote unit 110. The power is utilized to power the remote unit 110. The low data rate information may include configuration information or other information used for configuring the remote unit 110. The remote unit 110 communicates high data rate information (e.g., ultrasound image data), to the base unit 105.

In one exemplary embodiment, the power and low data rate information are communicated over a group of electrical conductors within the cable 115, such as copper wires. For example, a first electrical conductor may correspond to the ground terminal of a power supply. Other electrical conductors may communicate DC power to the remote unit 110. Yet other electrical conductors may communicate data for configuring the remote unit 110. For example, a pair of electrical conductors may correspond to an I²C data bus through which a processor of the remote unit 110 is configured.

High data rate information (e.g., higher than about 1 Gb/sec) may be communicated over one or more optical fibers. For example, the ultrasound image data collected by an ultrasound transducer may be communicated over the optical fibers. Optical signals communicated over the optical fibers may be converted to and from electrical signals via a converter circuit. The converter circuit may be positioned within the base unit 105, the handheld unit 110, or within a connector 120, 125 of the cable. In some implementations, a first converter circuit converts optical signals to electrical signals and is positioned in the base unit 105 or the connector 120 of the cable 115 that connects to the base unit. A second converter circuit converts electrical signals to optical signals and is positioned in either the remote unit 110 or a connector 125 of the cable 115 that is connected to the remote unit 110.

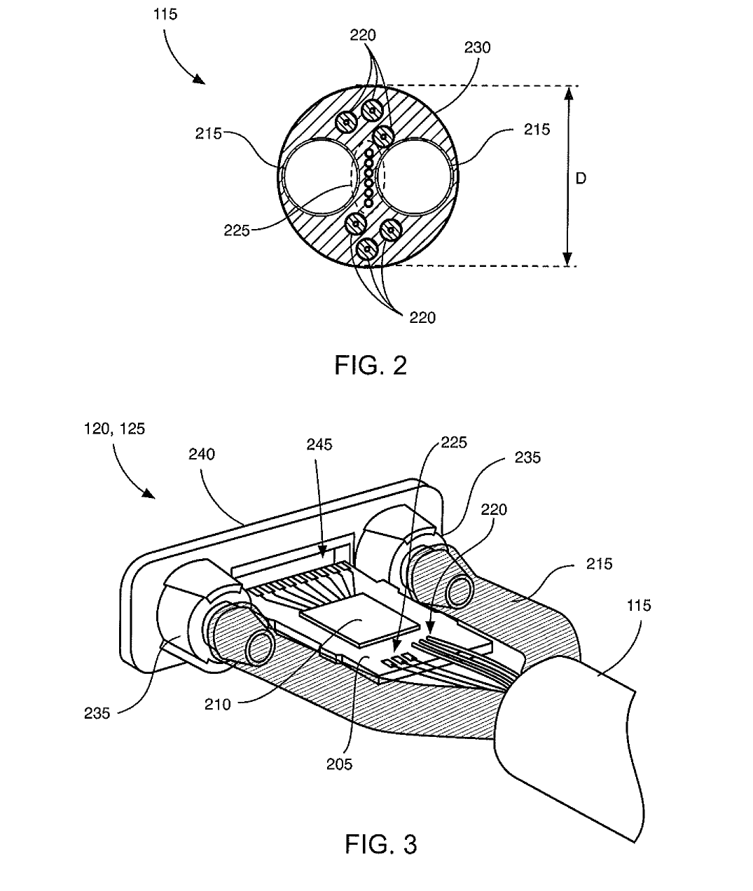

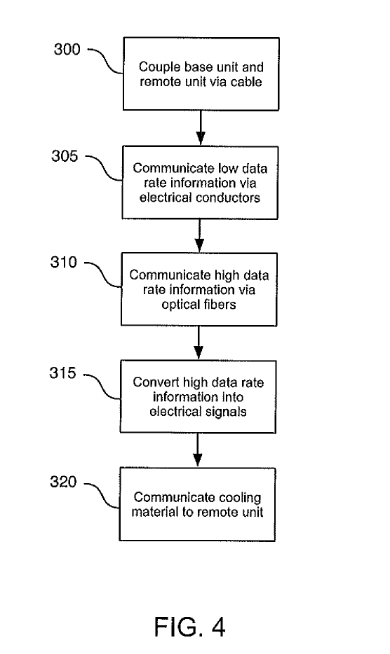

FIG. 2A illustrates a cross-section of an exemplary cable 115 that may couple the remote unit 110 to the base unit 105. The cable 115 includes a group of electrical conductors 225, a group of optical fibers 220, and an outer jacket 230. In some implementations, the cable 115 includes a pair of cooling tubes 215. As noted above, the electrical conductors 225 are utilized to communicate power and low data rate information such as configuration information from the base unit 105 to the remote unit 110. The electrical conductors 225 may correspond to wires in a range from about 28 gauge to 46 gauge solid or stranded wires. The number of electrical conductors 225 which may pass through the cable 115 depends on the requirements of the overall system, but is typically low (e.g. 15 or less).

The optical fibers 220 are utilized to communicate high data rate information from the remote unit 110 to the base unit 105. For example, image data from a transducer of an ultrasound machine may be converted into an optical signal and communicated over the optical fibers 220. The optical fibers 220 may be multimode optical fibers or single-mode optical fibers and may each have a diameter of between about 125 microns and 250 microns. Multimode optical fibers tend to have less stringent mechanical tolerances than single-mode optical fibers. On the other hand, single mode optical fibers tend to be smaller and have a higher bandwidth than multimode optical fibers. The number of optical fibers 220 will vary with the total data rate required, with each fiber typically carrying over 1 Gb/sec.

The cooling tubes 215 are configured to communicate a cooling material from the base unit 105 to the remote unit 110. The cooling material is utilized to cool components within the remote unit 110. The cooling material may be a liquid material or gas suitable for removing heat from electrical components. The cooling material may flow through a first cooling tube 215 towards the remote unit 105 where it will absorb heat generated at the remote unit 105. The heated cooling material will then flow back to the base unit 105 via a second cooling tube 215. The heated cooling material may then flow through a heat dissipation section located in the base unit 105 or connector 120 and then be returned to the remote unit 110. It should be noted that presence of cooling tubes is optional. For example, in some cases heat build-up is not an issue in the remote unit 110 thus obviating the need for cooling.

The jacket 230 is formed around the electrical conductors 225, optical fibers 220, and cooling tubes 215, if present. The jacket 230 may define a generally circular shape, as illustrated, or a different shape. The outside of the jacket 230 corresponds to the outer surface of the cable 115. The jacket 230 may also include an inner portion formed around the electrical conductors 225, optical fibers 220, and cooling tubes 215 so as to maintain the relative positions of these elements. In other implementations, the jacket 230 does not include an inner portion and the electrical conductors 225, optical fibers 220, and cooling tubes 215 are generally free to move within the jacket 230.

When cooling tubes 215 and optical fibers 220 are utilized in a cable 115, the diameter D of cable 115 may be comparable to typical ultrasonic imaging cables having only electrical conductors (about 0.33 inch (8.4 mm)). In implementations where cooling is not an issue and cooling tubes 215 may be eliminated from the cable 115, the diameter D of the cable 115 may be significantly smaller than typical ultrasonic imaging cables having only electrical conductors (about 0.25 inch (6.4 mm) or smaller). This is advantageous from an ergonomic perspective for handheld probes. It should also be understood that different applications may have different requirements, such as equipment-based solutions that would require higher data rates and potentially larger cable diameters.

FIG. 2B illustrates the interior portion of an exemplary connector 120,125 that may be utilized in connection with the cable 115 described above. The connector 120,125 is configured to mate to a complementary connector (not shown) provided on the base unit 105 and/or the remote unit 110. The exemplary connector 120,125 includes a circuit 205, a pair of cooling tube couplers 235, and a faceplate 240.

The cooling tube couplers 235 are configured to attach to the cooling tubes 215 described above. For example, the couplers 235 may be tapered to facilitate insertion of the couplers 235 within the cooling tubes 215. The couplers 235 may be friction fit to the cooling tubes 215 to prevent detachment of the cooling tubes 215 from the couplers 235. Alternatively or in addition, clamps and the like may be used to secure the cooling tubes 215 to the couplers 235.

The circuit 205 is configured to communicate power and information over the cable 115. For example, the circuit 205 may communicate power and control information to a remote unit 105 via the electrical conductors 225 in the cable 115. The circuit 205 may communicate high data rate information over the optical fibers 220 in the cable 115.

In some implementations, the circuit 225 includes a converter chip 210, such as a SPD2004 photodiode from Cosemi Technologies Inc., configured to convert data communicated over optical fibers 220 into electrical signals or vice versa that are subsequently communicated over conductive terminals 245. For example, when converting signals from optical to electrical, the converter chip 210 may de-multiplex the data communicated over one optical fiber 220 into a number of data channels that are communicated over a corresponding number of electrical conductors that are coupled to a corresponding number of conductive terminals 245. When converting from electrical to optical signals, the converter chip 210 may multiplex the electrical signals from the conductive terminals 245 into a single optical fiber 220.

In some implementations, a single converter chip 210 may convert to signals from electrical to optical and vise versa. This may facilitate the use of the same type of connector 120,125 on both ends of the cable 115. This in turn allows a given connector 120,125 of the cable to attach to either one of the base unit 105 and the remote unit 110. In other implementations, different converter chips 210 suited to one form of conversion or the other may be utilized. In this case, a given connector 120,125 may be key or configured to only connect to one or the other of the base unit 105 and the remote unit 110.

In other implementations, the converter chip 210 may be placed within the base unit 110 rather than in the connector 120,125. In this case, the size of the connector 120,125 may be reduced to save space.

FIG. 3 illustrates a group of operations for communicating between the base unit 110 and the remote unit 105 described above. One or more of these operations may be performed by the base unit 110 and/or the remote unit 105. In this regard, the base unit 110 and the remote unit 105 may include one or more non-transitory forms of storage media, such as RAMs, ROMs, and the like that store instruction code that is executable by a processor of one or both of the base unit 110 and the remote unit 105 to perform the operations described below.

At block 300, the base unit 110 and the remote unit 105 are coupled together via a cable, such as the cable 115, described above. The cable 115 may include one or more electrical conductors 225 for communicating electrical signals for communicating power and low data rate information between the base unit 110 and the remote unit 105. The cable 115 may also include one or more optical fibers 220 for communicating high data rate optical signals between the base unit 110 and the remote unit 105. The electrical conductors 225 and optical fibers 220 may be surrounded by an outer sleeve.

At block 305, low data rate information may be communicated over the electrical conductors 225 from the base unit 110 to the remote unit 105. For example, power for operating the remote unit 105 may be communicated over the electrical conductors 225. Control signals for configuring the remote unit 105 may be communicated over the electrical conductors 225. Other low data rate information may be communicated.

At block 310, high data rate information may be communicated over the optical fibers 220 from the remote unit 105 to the base unit 110. For example, image data information may be communicated from the remote unit 105 to the base unit 110 via the optical fibers 220. The high data rate information may be communicated synchronously or asynchronously with respect to the low data rate information communicated over the electrical conductors 225.

At block 315, the high data rate information communicated over the optical fibers 220 may be converted into electrical signals that are subsequently communicated over a group of conductive terminals 245. For example, a converter chip, such as the converter chip 210 described above may de-multiplex data communicated over the optical fibers optical fibers 220 into separate data channels and those signals may then be communicated over the conductive terminals 245. The converter chip 210 may be positioned within a circuit of a connector 120,125 for coupling the cable 115 to the base unit 110 or within the base unit 110 itself.

At block 320, in some implementations, cooling material may be communicated from the base unite 105 to the remote unit 110 to cool the remote unit 105. For example, the cooling material may flow continuously or on demand has the temperature of the remote unit 105 rises. Temperature information may be communicated from the remote unit 105 to the base unit 110 to facilitate this determination. The base unit 105 may process this information to determine whether to direct cooling material to the remote unit 110.

While various embodiments of the embodiments have been described, it will be apparent to those of ordinary skill in the art that many more embodiments and implementations are possible that are within the scope of the claims. For example, the shape of the connector may be varied. The connector may not include cooling tubes. The number of optical fibers and conductors in the cable may be increased or decreased to suit a particular bandwidth requirement. The various dimensions described above are merely exemplary and may be changed as necessary. Accordingly, it will be apparent to those of ordinary skill in the art that many more embodiments and implementations are possible that are within the scope of the claims. Therefore, the embodiments described are only provided to aid in understanding the claims and do not limit the scope of the claims.

What is claimed is:

1. A diagnostic system comprising:
a remote unit configured to gather information;
a base unit configured to process the gathered information; and
a cable that couples the remote unit to the base unit configured to carry the information, wherein the cable includes;
one or more electrical conductors for communicating electrical signals between the base unit and the remote unit;
one or more optical fibers over which the gathered information is communicated; and
an outside jacket formed around the one or more electrical conductors and the one or more optical fibers.

2. The diagnostic system according to claim 1, wherein the remote unit is an ultrasound transducer and the base unit is an ultrasound processor.

3. The diagnostic system according to claim 1, wherein a diameter of the cable is less than about 0.25 inches.

4. The diagnostic system according to claim 1, wherein a diameter of the one of the one or more optical fibers is between about 125 microns and 250 microns.

5. The diagnostic system according to claim 1, wherein the one or more optical fibers are multi-mode optical fibers.

6. The diagnostic system according to claim 1, wherein the cable includes a first connector positioned at a first end of the cable and a second connector positioned at a second end of the connector wherein the first connector includes a circuit configured to convert optical signals communicated over the one or more optical fibers into electrical signals.

7. The diagnostic system according to claim 6, wherein the second connector includes circuitry for converting electrical signals into optical signals.

8. The diagnostic system according to claim 1, wherein the base unit includes a circuit configured to convert optical signals communicated over the one or more optical fibers into electrical signals.

9. The diagnostic system according to claim 1, wherein the remote unit includes a circuit configured to convert optical signals communicated over the one or more optical fibers into electrical signals.

10. The diagnostic system according to claim 1, wherein the one or more conductors communicate power from the base unit to the remote unit.

11. The diagnostic system according to claim 1, wherein the one or more optical fibers communicate image data from the remote unit to the base unit.

12. A method for communicating information between a base unit and a remote unit of a diagnostic system, the method comprising:
coupling the base unit to the remote unit with a cable that includes:
one or more electrical conductors configured to communicate electrical signals between the base unit and the remote unit;
one or more optical fibers for communicating optical signals between the base unit and the remote unit; and
an outside sleeve formed around the one or more electrical conductors and that one or more optical fibers;
communicating first information from the base unit to the second unit over the one or more electrical conductors; and
communicating second information from the remote unit to the base unit over the one or more optical fibers.

13. The method according to claim 12, wherein the first information and the second information are communicated asynchronously.

14. The method according to claim 12, wherein the first information and the second information are communicated synchronously.

15. The method according to claim 12, wherein a data rate of information communicated over the one ore more electrical conductors is less than about 1 Gb/sec.

16. The method according to claim 12, wherein a data rate of information communicated over one of the one or more optical fibers is greater than 1 Gb/sec.

17. The method according to claim 12, wherein a diameter of the cable is less than about 0.25 inches.

18. The method according to claim 12, wherein the one or more optical fibers are multi-mode optical fibers.

19. The method according to claim 12, wherein the cable includes a first connector positioned at a first end of the cable and a second connector positioned at a second end of the connector, wherein the first connector includes a circuit configured to convert the optical signals communicated over the one or more optical fibers into electrical signals.

20. The method according to claim 19, wherein the second connector includes circuitry for converting electrical signals into optical signals.
